(19) United States
(12) Patent Application Publication
(10) Pub. No.: US 2020/0222222 A1
(43) Pub. Date: Jul. 16, 2020

(54) SYSTEMS AND METHODS FOR APPENDAGE SUPPORT

(71) Applicant: KENNESAW STATE UNIVERSITY RESEARCH AND SERVICE FOUNDATION, INC., KENNESAW, GA (US)

(72) Inventor: Simin NASSERI, Roswell, GA (US)

(21) Appl. No.: 16/742,520

(22) Filed: Jan. 14, 2020

Related U.S. Application Data
(60) Provisional application No. 62/792,099, filed on Jan. 14, 2019.

(51) Int. Cl. A61F 5/01; A61F 13/06

(52) U.S. Cl. CPC A61F 5/013; A61F 13/063; A61F 5/0127

(57) ABSTRACT

A support for an appendage includes a flexible main body having a generally hollow, tapered, cylindrical shape defining a cavity that is configured to receive an appendage of a wearer. One or more apertures are formed in a wall of the main body to provide bendability to the main body and to allow the appendage to breathe when disposed in the main body. The support further includes a support member disposed adjacent a surface of the main body opposite the one or more apertures to provide rigidity to the main body.

### CROSS-REFERENCED TO RELATED APPLICATIONS

This application is a non-provisional of and claims the benefit to U.S. Provisional Application No. 62/792,099 filed Jan. 14, 2019, which is hereby incorporated by reference in its entirety.

### BACKGROUND

Finger deformation is very common and has many causes and severity. Such deformities may include mallet finger, Dupuytren's contracture or disease, Boutonniere deformity, swan neck deformity, valgus deformity, clawfinger, etc. Table 1 describes different deformities, the cause of the deformities and their effect on fingers.

TABLE 1
Finger deformities, causes, description, and effects on finger.

| Name | Cause/Description | Effect on Fingers |
|---|---|---|
| Flexion | The flexor tendon becomes | Metacarpal phalangeal |
| deformity | inflamed and cannot move | (MCP), PIP, distal |
| | smoothly inside the sheath | interphalangeal (DIP) |
| | (trigger finger). | are bent sequentially. |
| Mallet Finger | Loss of the extensor | DIP bends specially in |
| | mechanism at the DIP | the ring finger |
| | joint. Also known as | (baseball finger). |
| | baseball finger or drop | |
| | finger. | |
| Dupuytren's | Hereditary disorder | The fingers will bend |
| contracture or | affecting mainly male | closer to the palm on |
| disease | patients. | the hand. |
| Boutonniere | Due to joint inflammatory | It causes the PIP joint |
| Deformity | diseases (RA or OA) or | to bend inward towards |
| | extensor mechanism | the palm (flexion) and |
| | rupture. | the DIP joint to bend |
| | | outward (extension). |
| Swan Neck | As above | PIP joint bends outward |
| Deformity | | and the DIP joint bends |
| | | inward towards the palm. |
| Valgus | RA or OA. Also, | The big toe will deform |
| deformity | Hallux Valgus caused by | and move in the direction |
| | using narrow-toe shoes. | of the other toes. |
| | Besides, abnormal | |
| | congenital joint surfaces | |
| | of the big toe. | |
| Clawfinger | Nerve damage caused by | The MCP joint bends |
| | diabetes or alcoholism, | outward and the other |
| | etc. leading to drop | joints bend inward. |
| | wrist or drop foot. | |

Finger deformity related to the conditions described in Table 1 may cause a noticeable curved or bent finger. A finger affected by one of these conditions may be unable to straighten on its own without splinting, physical therapy and other surgical and non-surgical methods. Each of conditions have a different root cause and severity varies based on the condition.

Most of the available finger supports are not resizable and comfortable, as they are typically made of rough fabrics/hook and loop material straps, or thermoset plastics without any flexibility. They are mainly designed to fully restrict the finger's motion when there has been any type of injury. However, for example, in the case of early stages of arthritis, more frequent or consistent wear is recommended. Also, available finger supports may not be adjusted considering variations in finger size and position. Furthermore, most of the available supports may not be configured for functional positions, and thus cannot be worn during normal life tasks.

Improvements are needed.

Similarly, foot deformities can have an effect on an individual's ability to perform daily tasks. For instance, some foot deformities substantially hinder an individual's ability to walk and perform everyday functions. The most common of these deformities globally is hallux valgus (HV), commonly known as bunions. HV is defined as an abnormal angulation of the great toe deviating laterally, with the first metatarsal deviating medially. This deviation is caused by tensile forces generated in the long flexor and the extensor tendon in the foot, causing the metatarsal head to move medially. This motion causes the great toe to move in toward the second and third toes. This applies pressure to the other toes and pressure on the side of the foot which causes discomfort and pain for the individual when walking. In fact, bunions are a deformity in portions of the feet known as the hallucial phalanges and medial prominence of the first metatarsal head the deformity is often associated with the symptoms of poor balance, foot pain, and overall decreased health quality of life. Walking pattern may be a leading cause of HV, as a typical walking pattern may place an oblique shear stress and axial torsion on the Flexor Hallucis Longus and Flexor Hallucis Brevis. This oblique shear stress and axial torsion due to propulsion of the human body results in displacement of hallucial sesamoids and the intervening Flexor Hallucis Longus tendon, thus causing bunions to form. As a result, most individuals who suffer from HV have trouble walking due to the displacement of the hallux or largest toe placing pressure on the joints resulting in pain for the individual during movement.

The treatment options for HV generally fall into two categories surgical and non-surgical. The current surgical method of fully correcting deformity of the Hallux Valgus is an intrusive surgery results in and extensive recovery and is very costly and may be out of reach for many individuals. Accordingly, more conservative non-surgical methods have been developed.

Some non-surgical approaches which are more conservative are proper fitting footwear that have a wide and deep toe box, kinesiology tape, non-steroidal anti-inflammatory drugs (NSAIDs), and muscle relaxants. In addition to these there exists a wide variety of orthotic options to treat HV such as insoles, night splints, and toe separators and supports. Within the toe separator category there are cheaper prefabricated silicon toe separators (TS) on the market, but these boast lower effectiveness when compared to surgery. The prefabricated silicon TS also have a lower compliance rate because they are not custom to the individual and thus they often do not fit the individuals foot very well and this leads to some discomfort which leads to patients being less likely to wear them.

Improvements are needed.

### SUMMARY

The present disclosure relates to composite support devices for appendages such as finger and foot. The support devices may be formed from a flexible, durable polymer material and may further comprise rigid supports inserted into one or more regions of the device. The supports may be fabricated to tolerate applied forces without any permanent deformation, allow for size and position adjustments, and may be used for patients who already have distorted appendages and are working to regain some appendage functions.

One aspect of the present disclosure relates to a support for an appendage including a flexible main body having a generally hollow, tapered, cylindrical shape defining a cavity that is configured to receive an appendage of a wearer. One or more apertures are formed in a wall of the main body to provide bendability to the main body and to allow the appendage to breathe when disposed in the main body. The support may also include a support member disposed adjacent a surface of the main body opposite the one or more apertures to provide rigidity to the main body. In such embodiments, the support may be a finger support.

Another aspect of the present disclosure relates to support for an appendage, the support including a flexible main body further including one or more first receptacles disposed adjacent a first end of the main body and a second receptacle disposed adjacent a second end of the main body opposite the first end. The main body may further include a member coupled between the one or more first receptacles and the second receptacle, and a support member disposed adjacent the member of the main body to provide rigidity to the main body. In such embodiments, the support may be a foot support.

The embodiments disclosed herein allow wear of the support at any stage of deformity to allow for the correction process to begin. Such embodiments also allow for simple manufacturing and changes to be made as the wearer's recovery progresses.

Methods for forming the supports described herein are also contemplated. Exemplary embodiments of the supports described herein may be fabricated using additive manufacturing (e.g., 3D printing) techniques, extrusion techniques, compression molding or like manufacturing processes.

### BRIEF DESCRIPTION OF THE DRAWINGS

The following drawings show generally, by way of example, but not by way of limitation, various examples discussed in the present disclosure. In the drawings:

FIG. 1 shows an example finger support according to embodiments of the disclosure.

FIG. 2 shows an example finger support including a rigid support member.

FIGS. 3A-3C show alternative examples of a finger support according to embodiments of the disclosure.

FIGS. 4A-4D show further alternative examples of a finger support according to embodiments of the disclosure.

FIGS. 5A-5B show alternative examples of a finger support according to embodiments of the disclosure.

FIGS. 6A-6B show alternative examples of a finger support according to embodiments of the disclosure

FIG. 7 shows an example foot support according to embodiments of the disclosure.

FIG. 8 shows an example processing step for the example foot support according to embodiments of the disclosure.

FIG. 9 shows an alternative view of example foot support according to embodiments of the disclosure.

FIG. 10 shows an alternative example foot support according to embodiments of the disclosure.

### DETAILED DESCRIPTION

In certain aspects, supports for appendages such as fingers and feet are shown and described.

Embodiments of the present disclosure provide a finger support that enables the wearer to perform daily functions, while providing comfort and flexibility. The finger support would be beneficial for patients with arthritis who have finger deformity. A finger support according to embodiments of the disclosure would be configured to be worn such that a finger subject to any of these conditions may take the shape of the support gradually. This type of device and treatment will slow the progression of deformity without surgery or other costly treatments, and in some cases, correct the finger deformity and improve its function.

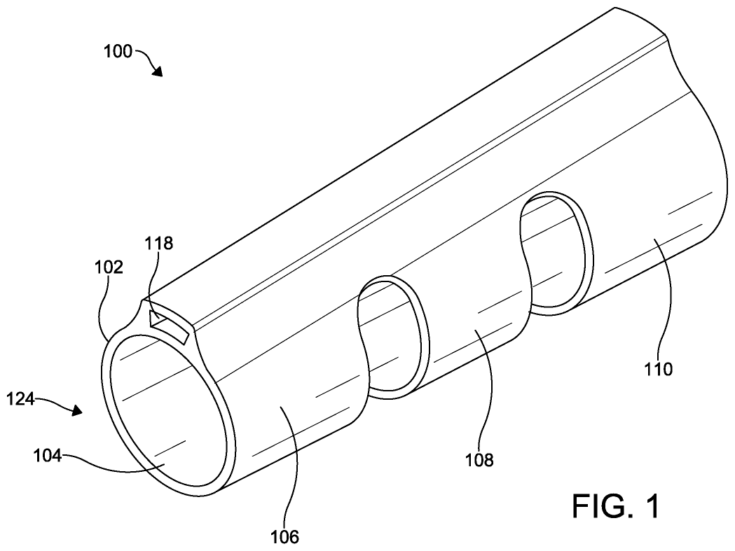

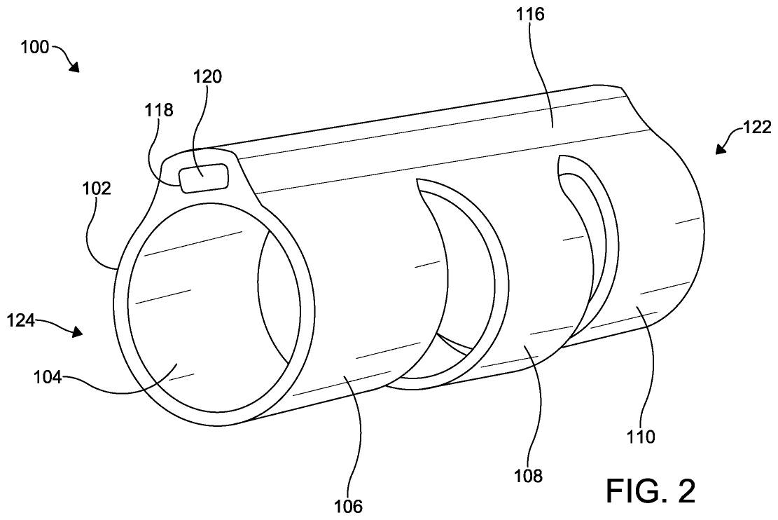

FIG. 1 illustrates an exemplary finger support 100 according to embodiments of the disclosure. The finger support 100 may help relieve pain from inflammation and allow the finger to straighten over time. The finger support 100 may be worn during resting (e.g., with no applied load) and functional positions (e.g., performing normal life tasks). The finger support may include a flexible main body 102 having a generally hollow, tapered, cylindrical shape defining a cavity 104 that is configured to receive an appendage of a wearer. One or more alternating flexible rings 106, 108, 110 and apertures 112, 114 are formed in a wall of the main body 102 to provide bendability to the main body and to allow the appendage to breathe when disposed in the main body. The rings 106, 108, 110 may be configured to wrap around an appendage (e.g., a finger). The support 100 may also include a support member 116 disposed adjacent a surface of the main body 104 opposite the one or more rings 106, 108, 110 and apertures 112, 114 to provide rigidity to the main body. Support member 116 may be a longitudinal spine substantially running the length of the main body 102 and may further include a channel 118 that runs the length of the support member 116. As used herein, longitudinal may refer to running lengthwise from a first end of a cylindrical support (e.g., finger support 102) to a second end, as opposed to following a circumference of the cylindrical support. The channel 118 may be configured to receive a rigid support 120, as shown in FIG. 2, which illustrates the finger support 100 including a rigid support 120 inserted into the channel 118.

The finger support 100 may be tapered such that a first end 122 has a diameter greater than a second end 124 diameter, substantially following the shape of a finger. The finger support 100 may be internally only, such that the outer diameter of the main body is the same across the length of the finger support 100. Alternatively, the main body may be tapered internally and externally, such that the ratio between the inner diameter and the outer diameter of the main body remains the same at any given point along the length of the main body. The finger support 100 may include one or more bevels to reduce stress concentrations at specific points or regions of an appendage when the finger support 100, such as within internal cavities or adjacent the flexible rings 106, 108, 110. The finger support 100 is configured to be durable and rigid, capable of holding an amount of force of a finger when bending. The finger support 100 may include one or more indents between the flexible rings 106, 108, 110 to provide bending of the finger support 100 and to accommodate any bend angle. Support member 116 may be configured to align with an underside region of a finger (e.g., the “palm side”). Rigid support 120 may be, for example a column or sheet of metal such as aluminum or carbon fiber. Because of its flexibility, the finger support 100 may accommodate small deformations while withstanding stretch fatigue. Finger support 100 allows for size and finger position adjustments, and may be used with distorted fingers, e.g., for users working to regain some finger function. In some embodiments, finger support 100 may be approximately 2.25 in in length, but the length may vary to accommodate finger length of a wearer. Finger support 100 may provide various bend angles and a friction-hold grip around the finger.

Methods for forming the finger support 100 may include one or more first fabrication processes. Fabrication processes for creating finger support 100 may include extrusion, additive manufacturing (or 3D printing), and compression molding. For instance, finger support 100 may be fabricated using a 3D PolyJet printer. Elastomers (e.g., natural rubber, synthetic rubber, and thermoplastic elastomers) that provide sufficient elasticity and extensibility may be utilized or forming at least a portion of the main body 102 and support member 116 of the finger support 100. Advantageously, elastomers used to form finger support 100 components may be soft to the touch and reduce or minimize skin irritation for a wearer, and provide a comfortable fit around the finger that is resistant to tearing. In exemplary embodiments, a tear resistant polymer, such as DuraForm® Flex is utilized. Other materials may be used, including, but not limited to, TangoBlack, TangoGray, or Agilus 30 FLX, and other DuraForm® polymers. Agilus30-FLX has high tensile tear resistance (5-7 kg-cm⁻¹). The Agilus30 family of polymers provides superior tear-resistance, elongation at break and rubber-like texture. In some embodiments, a fiber reinforced polymer (FRP) material may be used.

During fabrication, a support resin material may be deposited within one or more cavities of the finger support 100. For example, the support resin material may be Fullcure 705 or the like. Methods for fabrication finger support 100 therefore include removing the resin. In some instances, the resin may be removed by soaking the finger support 100 device in a 2% sodium hydroxide solution for a set amount of time (e.g., one to two hours), with subsequent submergences for approximately 5 minutes if needed to aid removal process. The resin may be removed manually and then submerged in a sodium hydroxide solution (e.g., for approximately 5 minutes), repeated as necessary.

In some embodiments, finger support 100 may be a composite device composed with metal fibers, which may impart greater strength onto the finger support 100 with or without the inserted strips of metal or carbon fiber. For instance, a Connex 3D printer capable of printing with multi-material may be utilized to form a composite finger support 100.

The support is soft and comfortable, yet very strong to tolerate a range of loads. When finger support 100 is worn, an amount of force may be applied by a finger to the aluminum or carbon fiber sheet. The larger the finger, the greater the force that may be exerted on the tendon. While finger force varies across different body types and between sexes, an average flexion force for a finger may be in the range of 1-1.25 N. In some instances, average flexion force may be 1.17 N, and the greatest flexion force may be 1.21 N, corresponding to high expression level performance conditions. Accordingly, finger support 100 is configured to withstand these applied forces.

As mentioned above, support 100 may be a composite device formed from a combination of flexible polymer and metal or carbon fiber. In exemplary embodiments, support 100 may be formed using either a combination of DuraForm® Flex and Aluminum 6061-T6 (SS) or DuraForm® Flex and carbon fiber. Results of stress and displacement tests for these combinations are shown below in Table 2:

TABLE 2
Stress and Displacement Results.

| Material | von Mises Stress (MPa) | Displacement |
|---|---|---|
| DuraForm ® Flex and | 52.90 | 0.9217 |
| Aluminum 6061-T6 (SS) | | |
| DuraForm ® Flex and | 55.66 | 0.2822 |
| Carbon Fiber | | |

Finger support 100 provides support and alignment for joints, especially painful joints. Finger support 100 may gradually straighten a finger and/or correct abnormal curvatures. Finger support 100 is designed for the resting position to ease pain and inflammation during a painful flare-up or a period of unusual discomfort. In other embodiments, finger support 100 may modify a severely bent fingers to provide one or more functional or working position of the finger or multiple fingers (e.g., when fingers and thumb are in flexion) thereby reducing pain associated with certain tasks. Advantageously, finger support 100 may be worn during routine and daily tasks. Finger support 100 may be resizable and modified to be used in functional finger position.

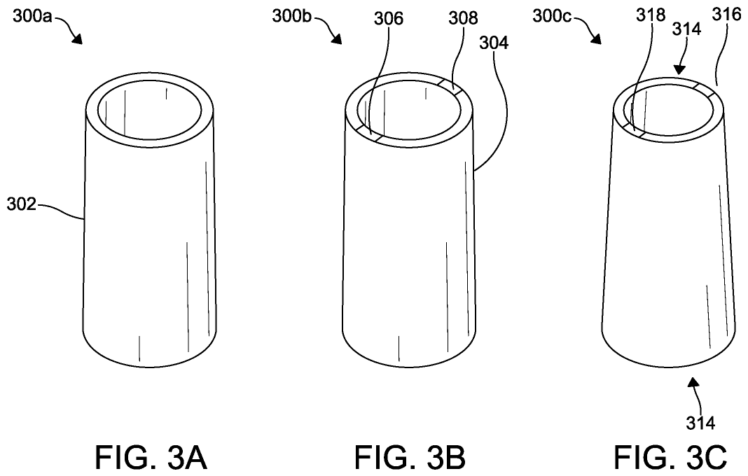

FIG. 3A illustrates a substantially cylindrical example of an alternative finger support 300a including a flexible cylindrical body 302 metal fibers incorporated in to the polymer material. In such an embodiment, a multi-material 3D printer may be used to incorporate metal fibers. Alternatively, the composite material may be extruded. FIG. 3B illustrates a substantially cylindrical example of an alternative finger support 300b including cylindrical body 304 and a plurality of longitudinal slots 306, 308 disposed on opposite sides of the cylindrical body 304 configured to receive one or more support strips (e.g., strips of aluminum metal), thereby configured to accept additional force associated with a bending finger. FIG. 3C illustrates an example of an alternative tapered cylindrical finger support 300c, which includes a flexibly tapered cylindrical body 310 configured with a first end 312 having a first diameter which tapers to a second end 314 having a second diameter, the second diameter being smaller than the first diameter, to accommodate a finger taper. Finger support 300c may also include a plurality of longitudinal slots 316, 318 disposed on opposite sides of the cylindrical body 310 configured to receive one or more support strips (e.g., strips of aluminum metal), thereby configured to accept additional force associated with a bending finger. Each of the aforementioned alternative finger supports, 300a, 300b and 300c may be formed using any of the fabrication methods or processes described herein. In some instances, an extrusion process applying a heat treatment methods to the cylindrical body may be used to form alternative finger supports, 300a, 300b and 300c.

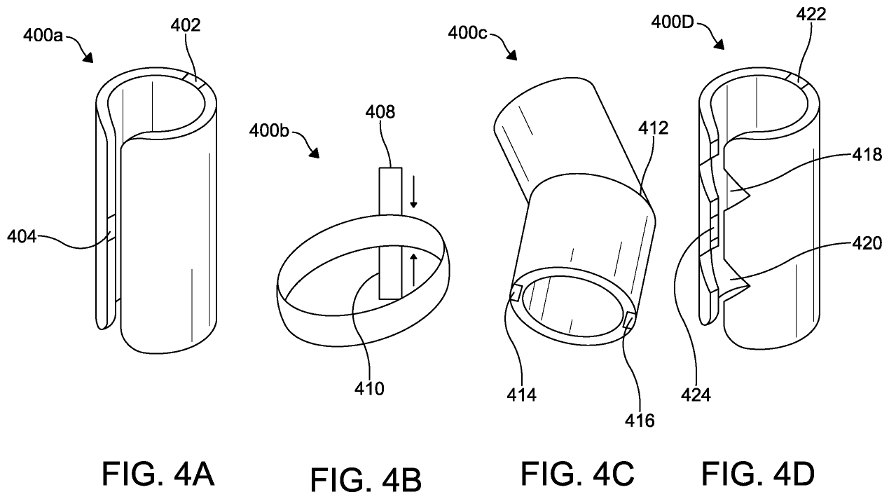

FIGS. 4A-4D illustrate further alternative embodiments of finger support 100. FIG. 4A illustrates a substantially cylindrical example of an alternative finger support 400a that accommodates the opening and closing of the support, hence the user will be able to adjust the finger support to fit the size of their fingers or make only the tip of the support narrower.

FIG. 4B illustrates a metal support insert 400b configured to be inserted into finger support 400a. Finger support 400a includes at least two slots, one longitudinal slot 402 and one horizontal slot 404 configured to receive metal support insert 400b. Metal support insert 400b may include a horizontal support ring 406, and one or more longitudinal support strips 404, 410. Longitudinal slot 402 and horizontal slot 404 may be configured with dimensions that allow insertion of one or more of the support ring 406 and strips 408, 410. In embodiments, one each of the two longitudinal support strips 408, 410 may be inserted from top and bottom into the longitudinal slot 402 after the horizontal support ring 406 is inserted. Alternatively, a support sheet (not shown) may be inserted longitudinally. In exemplary embodiments, the support strip and sheet components are metal. The longitudinal sheet dimensions may be approximately 4.74 mm by 1.60 mm by 50.40 mm. The circumference of the metal sheet may be substantially equal to the circumference of the support (e.g., approximately 4.74 mm). Bevels may be formed in certain regions of the finger support 400a to lower stress concentration in corners. The interior section of the rings may be rounded to avoid tearing when bending.

As with finger support 100, circular hollow region or cavities of the finger support 400a may be formed during the 3D printing fabrication process, which may use a resin material to fill these cavities during printing. The finger support 400a may include a radial cut which allows the resin to be removed, but is configured to retain the aluminum rings. Longitudinal support strips 408, 410 may rest above one or more horizontal support rings 406 while remaining in contact with the support rings. The longitudinal support strips 408, 410 may also be curved for improved contact between the support sheet and the finger support. Finger support 400a is configured to adaptable in design, thereby suitable for various functional position of the finger easily.

FIG. 4C illustrates an example of an alternative finger support 400c ideal for patients that are unable to fully extend their fingers in functional positions. Finger support 400c may include one or more bends 412 to accommodate a bent finger. Depending on the severity of the finger's deformity, finger support 400c may be formed at various angles to fit a finger at its maximum possible angle of extension. If a patient is attempting to strengthen the finger to eventually be fully extended again, the patient can “train” the finger by changing the support as often as directed to increase their finger's angle of extension each time the support is changed. Aluminum, steel, or carbon-fiber support strips may still be inserted on either side of the finger support 400c. In some instances, finger support 400c includes a plurality of slots 414, 416 configured to receive one or more angled support strips, that, when inserted provide adequate strength to the bent finger support 400c.

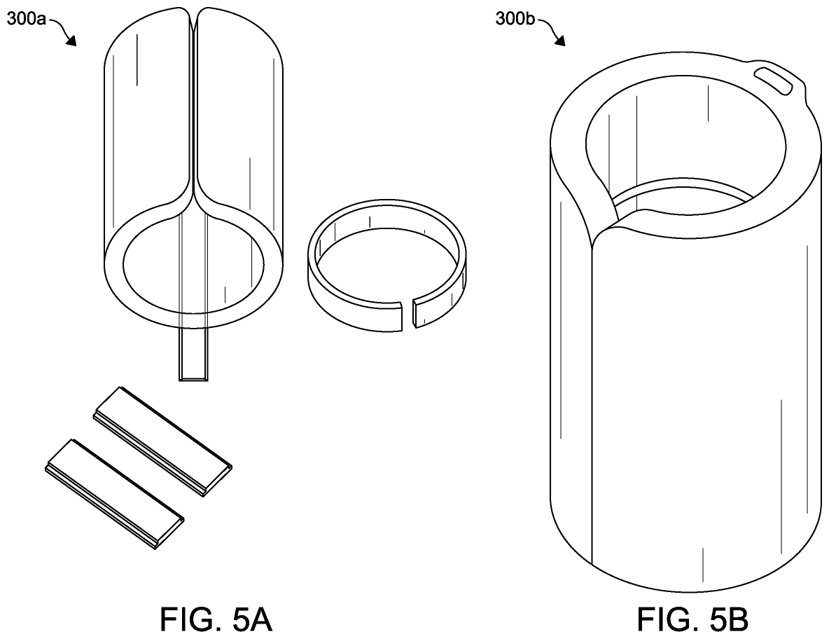

FIG. 4D illustrates an alternative substantially cylindrical example of a finger support 400d. Finger support 400d may be fully flexible, and configured to be adjusted for various bend angles. To this end, finger support 400d may include a plurality of slits disposed on a the back side of the support (not shown) and a plurality of triangle shaped cuts (e.g., cuts 418 and 420) in the front where the support device 400d opens and closes. The slits provide additional flexibility, such that the support device 400d is bendable and configured to adjust to more positions. In some embodiments, finger support 400d slits and cuts may be manufactured as it is flatted out and then shaped as a cylinder with the aid of a horizontal support insert. Finger support 400d includes at least two slots, one longitudinal slot 422 and one horizontal slot 424 configured to receive metal support insert 400b. Horizontal support insert may be metal. Springback of the polymer may be resolved by heat treatment and recrystallization. FIGS. 5A and 5B illustrate fabricated finger support 300a including the support strips and support rings.

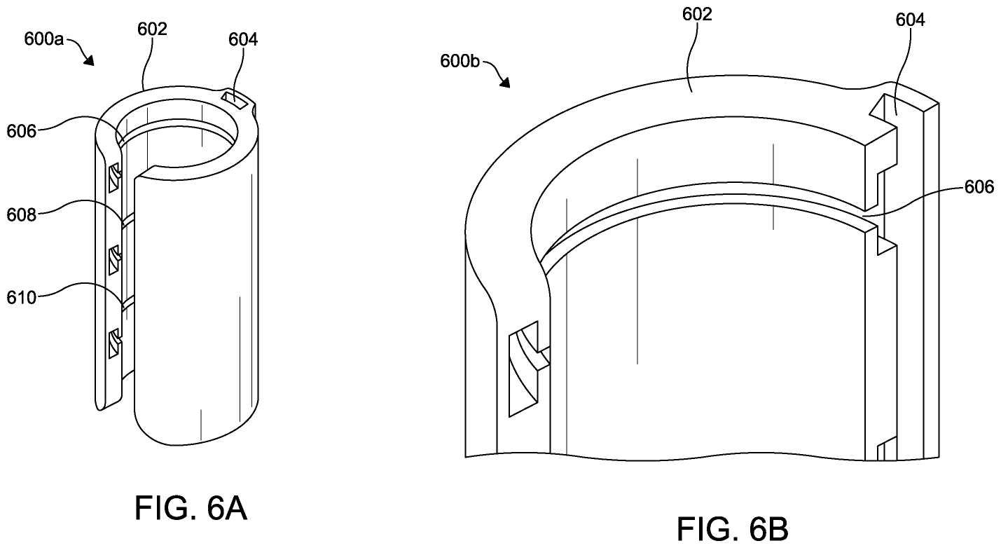

FIG. 6A illustrates a further alternative embodiment finger support 600a to finger support 300a. Finger support 600a may be a composite model formed from a durable polymer. Finger support 600a may include a flexible main body 602 and may be configured with at least one longitudinal slot 604 configured to receive one or more longitudinal support strips (not shown) and a plurality of horizontal slots 606, 608, 610 and/or cutouts configured to receive a plurality of support rings (not shown). Support strips and rings may be formed from metal or carbon fiber. FIG. 6B is an exploded version 600b of a portion of finger support 600a illustrated in FIG. 6A.

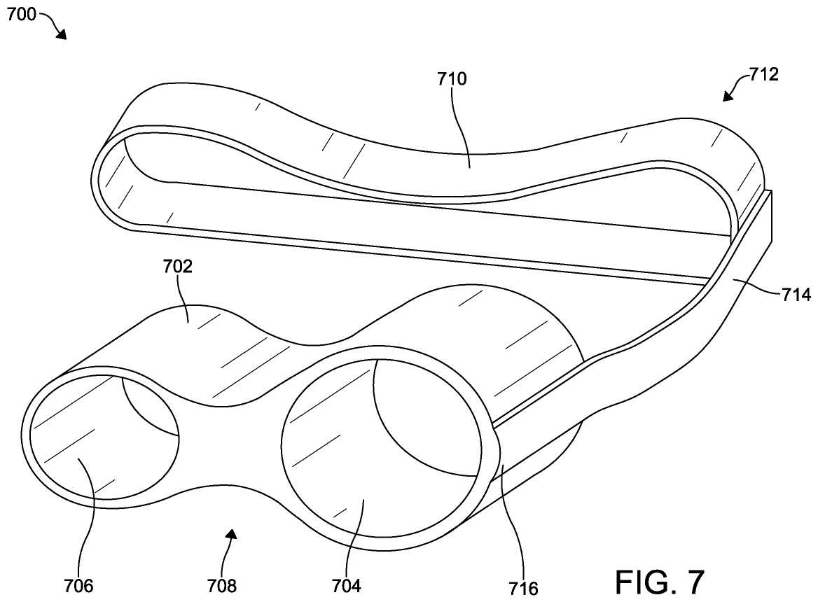

In further embodiments, a foot support is contemplated. FIG. 7 illustrates a foot support 700 according to embodiments of the disclosure. Foot support 700 may address a condition known as hallux valgus, commonly referred to as bunions. Foot support 700 may include a flexible main body 702. Main body 702 may include one or more first receptacles 704, 706 disposed adjacent a first end 708 of the main body and a second receptacle 710 disposed adjacent a second end 712 of the main body 702 opposite the first end 708. First receptacles 704, 706 may be thin polymeric rings or shells configured to wrap around or encircle and substantially separate first and second toes of a foot. First receptacles 704, 706 may provide a friction-hold grip around the toes. Second receptacle 710 may be configured to wrap around another portion of a foot (e.g., a midsection or heel section of a foot). Second receptacle 710 may provide a friction-hold grip around the foot portion that it encircles.

The main body 702 may further comprise a flexible member 714 coupled between the one or more first receptacles 704, 706 and the second receptacle 710 and may include a channel or slot 716 configured to receive a rigid support member that may be disposed within the flexible member 714 of to provide rigidity to a portion of the main body 702 configured to be worn adjacent a medial portion of a foot. Rigid support member is configured to maintain alignment of the first toe (e.g., to keep the large toe substantially straight). Foot support 700 is configured such that, when worn, the encircled toes remain substantially parallel while maintaining a predetermined spacing between the toes.

Foot support 700 configured to allow the support to be worn within a shoe and allows for daily use. The second receptacle 710 (e.g., “wrap”) around the foot provides a location for a reaction force opposing the bunion. This reaction helps to correct the deformity by pulling the toes in the opposite direction of the deformity. The first receptacles 704, 706 are configured to wrap around the toes to allow for the correction of both the largest toe and the toe beside it which are often affected by the hallux valgus deformity. The area wrapping the toes also has a space that presses the two toes wrapped into opposing directions to correct deformity in the two toes associated with bunions. As with the finger support 100, the foot support 700 may be constructed using a combination of a polymer material and metal or carbon fiber inserts to provide corrective support. Namely, the flexible components of the foot support 702 may be constructed of a polymer based material and the rigid support member (e.g., solid support beam) used to correct the deformity may be formed from a rigid material such as a metal or carbon fiber. The support may be low profile and designed for maximum mobility and correction for the wearer.

The use of the support for correction will allow the wearer to wear the device daily and provides a noninvasive method to correct the deformity. Foot support 700 allows the patient to go about daily activities while wearing the brace and still be fully mobile. The design and materials used to form the foot support 700 may be selected with patient mobility as a priority. The polymer material that surrounds the toes allows for a comfortable fit that can be worn for extended periods of time. The foot support 700 corrects the deformity by applying a slight pressure in the opposite direction of the curvature resulting in the gradual straightening of the joint. The use of the rigid support member (e.g., rigid support beam or insert) allows the soft polymer to be used for comfort and the rigid support member provides the rigidity needed to correct the deformity. The polymer surrounding the toe may be connected to the second receptacle 710 (e.g., a strap or wrap) that surrounds the foot and prevents the support from twisting while being worn. The combination of these design features allow for effective correction of the deformities with patient use and comfort as a priority.

The force of the toe deformity is applied to the beam that is inserted into the support. The beam acts as a cantilever beam and correct equilibrium equations can be applied to the beam and forces can be found and simulated. In some instances, a hallux valgus deformity may have an angle ranging from 15 to 45 degrees. A force of 2 Newtons inward on the toes may be applied and a force of 1 newton may be applied to the rigid support member where the side of the foot is in contact with the support. The deformity may causes the toes to put forces on the rigid support member inserted into the flexible member 714, resulting in a cantilever beam with distributed forces acting along the length of the rigid support member and foot. The rigid support member dimensions may be small to allow the patient to wear the support inside footwear of their choice.

The foot support 700 may be constructed using similar fabrication process as those described above with respect to finger support 100. Accordingly, methods for forming the foot support 700 may include one or more first fabrication processes. Fabrication processes for creating foot support 700 may include extrusion, additive manufacturing (or 3D printing), and compression molding. For instance, foot support 700 may be fabricated using a 3D PolyJet printer. The materials used to form foot support 700 may be selected based on desired material properties such as weight and elastic modulus. Elastomers (e.g., natural rubber, synthetic rubber, and thermoplastic elastomers) that provide sufficient elasticity and extensibility may be utilized or forming at least a portion of the flexible main body 702 and flexible member 714 of the foot support 700. Advantageously, elastomers used to form foot support 700 components may be soft to the touch and reduce or minimize skin irritation for a wearer, and provide a comfortable fit around the finger that is resistant to tearing. In exemplary embodiments, a tear resistant polymer, such as DuraForm® Flex is utilized. Other materials may be used, including, but not limited to, TangoBlack, TangoGray, or Agilus 30 FLX, and other DuraForm® polymers. Agilus30-FLX has high tensile tear resistance (5-7 kg-cm⁻¹). The Agilus30 family of polymers provides superior tear-resistance, elongation at break and rubber-like texture. In some embodiments, a fiber reinforced polymer (FRP) material may be used. A support resin (e.g., Fullcure 705) may be utilized during the printing process.

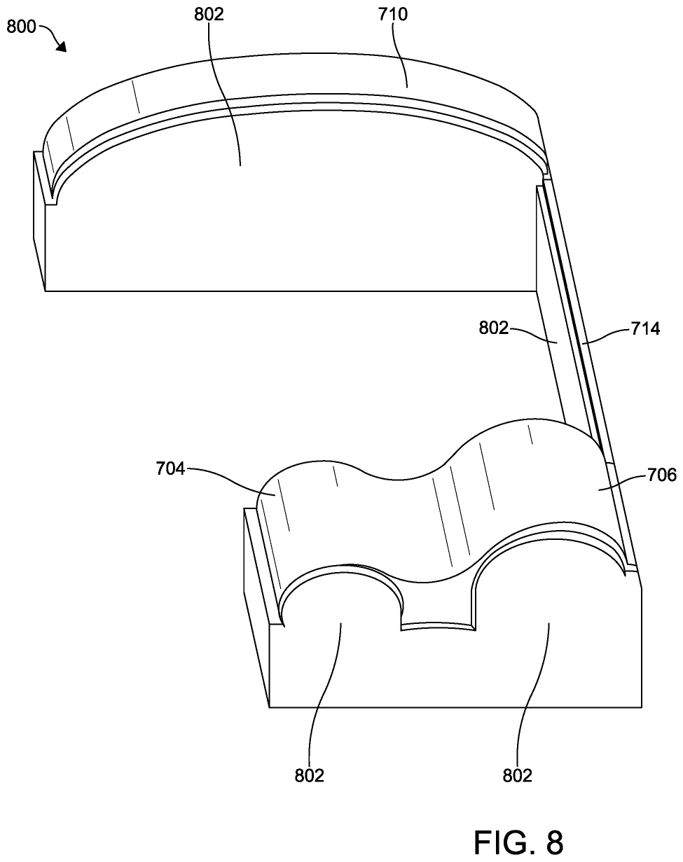

Additive manufacturing or 3D printing allows for a fully supported print, enabling printing of a flexible foot support main body. During fabrication, a support resin material may be deposited within one or more regions of the foot support 700. FIG. 8 shows an example processing step for the example foot support according to embodiments of the disclosure. FIG. 8 illustrates an exemplary foot support 700 prior to the support material being removed. As shown in FIG. 8, after printing, support resin material 802 may be present within certain regions of the foot support 700 (e.g., within first receptacles 704, 706, second receptacle 710 and flexible member 714). The support resin material 802 may be Fullcure 705 or the like. Methods for fabrication of a foot support 700 therefore include removing the resin. To accomplish resin removal, a foot support 700 comprising support resin material within the first receptacles 704, 706, second receptacle 710 and flexible member 714 may be placed into a pressure washing chamber and the support resin material 802 may be removed with water The result of the pressure washer chamber may be a bare flexible foot support 700 that may be rinsed with low pressure water and configured for insertion of a rigid support member into flexible member 714.

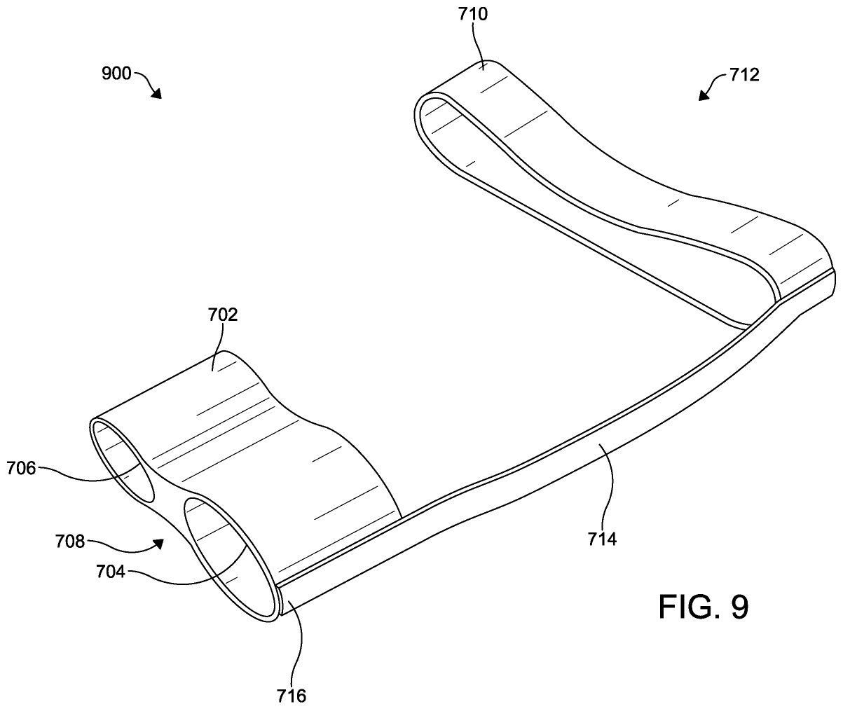

FIG. 9 illustrates the foot support 900 after fabrication and removal of the resin support material. As shown in FIG. 9, the foot support 900 is configured to receive a rigid support member (e.g., a metal or carbon fiber rod) within the channel 716 beginning at the first receptacle 704 and extending to the second receptacle 710 (e.g., foot strap) of the foot support 900.

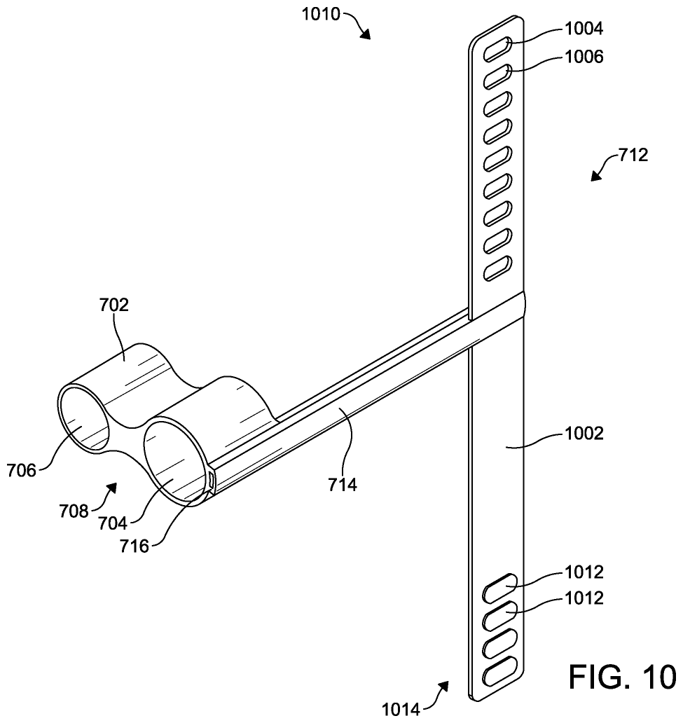

FIG. 10 shows an alternative example foot support 1000 according to embodiments of the disclosure. As with foot support 700, foot support 100 includes a flexible main body 702. Main body 702 may include one or more first receptacles 704, 706 disposed adjacent a first end 708 of the main body 702. Foot support 1000 may further include an adjustable strap 1002 disposed at a second end 712 of the main body 702. Adjustable strap 1002 may comprise an adjustable closure, configured to be adjusted to a plurality of foot sizes. Adjustable strap 1002 may include a plurality of apertures 1004, 1006, on a first end 1010, and a closure mechanism 1012 disposed on a second end 1014. The main body 702 may further comprise a flexible member 714 coupled between the one or more first receptacles 704, 706 and the adjustable strap 1002 and may include a channel or slot 716 configured to receive a rigid support member that may be disposed within the flexible member 714 of to provide rigidity to a portion of the main body 702 configured to be worn adjacent a medial portion of a foot. Foot support 1000 may also be configured to accommodate various foot sizes easily.

In some embodiments, foot support devices described herein may be a composite devices composed with metal or carbon fibers mixed with a polymer material, which may impart greater strength onto the foot support 700 with or without the inserted strips of metal or carbon fiber. For instance, a Connex 3D printer capable of printing with multi-material may be utilized to form a composite foot support 700.

The foot supports described herein are designed to be flexible enough to be worn daily and provide correction over a period of time without the need for surgery while also providing comfort and functionality. The support may also aid with inflammation and relieve pain during moments of discomfort. The foot supports may allow for wear of the device at any stage of a foot deformity to allow for the correction process to begin. The foot supports may also allow for simple manufacturing and changes to be made as the patients recovery progresses.

Various combinations of elements of this disclosure are encompassed by this disclosure, e.g., combinations of elements from dependent claims that depend upon the same independent claim.

Throughout the description and claims of this specification, the word “comprise” and variations of the word, such as “comprising” and “comprises,” means “including but not limited to,” and is not intended to exclude, for example, other components, integers or steps. “Exemplary” means “an example of” and is not intended to convey an indication of a preferred or ideal embodiment. “Such as” is not used in a restrictive sense, but for explanatory purposes.

It is to be understood that the methods and systems are not limited to specific methods, specific components, or to particular implementations. It is also to be understood that the terminology used herein is for the purpose of describing particular embodiments only and is not intended to be limiting. Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the disclosure should be determined with reference to the appended claims, along with the full scope of equivalents to which such claims are entitled.

What is claimed is:

1. A support for an appendage, the support comprising:
a flexible main body having a generally hollow, tapered, cylindrical shape defining a cavity that is configured to receive an appendage of a wearer, wherein one or more apertures are formed in a wall of the main body to provide bendability to the main body and to allow the appendage to breathe when disposed in the main body; and
a support member disposed adjacent a surface of the main body opposite the one or more apertures configured to receive a rigid support member.

2. The support of claim 1, wherein the flexible main body comprises a plurality of flexible rings, each of the plurality of flexible rings adjacent at least one of the one or more apertures.

3. The support of claim 1, wherein the support member comprises a longitudinal spine substantially running the length of the flexible main body.

4. The support of claim 3, wherein the longitudinal spine includes a channel substantially running the length of the longitudinal spine.

5. The support of claim 4, wherein the channel is configured to receive the rigid support member.

6. The support of claim 1, wherein the flexible main body comprises a polymer.

7. The support of claim 1, wherein the support member comprises a metal.

8. The support of claim 1, wherein the support member comprises carbon fiber.

9. A support for an appendage, the support comprising:
a main body having a generally hollow cylindrical shape defining a cavity that is configured to receive an appendage of a wearer; and
a support member disposed adjacent a surface of the main body opposite.

10. The support of claim 9, wherein the support member comprises a longitudinal spine substantially running the length of the flexible main body.

11. The support of claim 10, wherein the longitudinal spine includes a channel substantially running the length of the longitudinal spine.

12. The support of claim 11, further comprising a rigid member configured to be inserted into the channel.

13. The support of claim 9, wherein the flexible main body comprises a polymer.

14. The support of claim 9, wherein the support member comprises a metal.

15. The support of claim 9, wherein the support member comprises carbon fiber.

16. A support for an appendage, the support comprising:
a flexible main body comprising one or more first receptacles disposed adjacent a first end of the main body and a second receptacle disposed adjacent a second end of the main body opposite the first end, the main body further comprising a flexible member coupled between the one or more first receptacles and the second receptacle; and
a support member disposed within the flexible member of the main body to provide rigidity to the main body.

17. The support of claim 16, wherein the flexible main body comprises a polymer.

18. The support of claim 16, wherein the support member comprises a metal.

19. The support of claim 16, wherein the support member comprises carbon fiber.

20. The support of claim 16, wherein the support member is at least partially embedded in the main body.
